(19) United States
(12) Patent Application Publication
(10) Pub. No.: US 2017/0360849 A1
(43) Pub. Date: Dec. 21, 2017

(54) BACTERIA DONORS AND PHARMACEUTICAL COMPOSITIONS

(71) Applicant: Z.M.D. MEDICAL LTD., Netivot (IL)

(72) Inventor: Mark GAIDES, Ashdod (IL)

(73) Assignee: Z.M.D. MEDICAL LTD., Netivot (IL)

(21) Appl. No.: 15/525,475

(22) PCT Filed: Nov. 17, 2015
(86) PCT No.: PCT/IL2015/051106

§ 371 Date: May 9, 2017

Related U.S. Application Data
(60) Provisional application No. 62/081,032, filed on Nov. 18, 2014.
(60) Provisional application No. 62/081,037, filed on Nov. 18, 2014.
(60) Provisional application No. 62/081,034, filed on Nov. 18, 2014.

(51) Int. Cl. A61K 35/741; G01N 33/50; C12N 1/20; A61K 35/00

(52) U.S. Cl. CPC A61K 35/741; C12N 1/20; G01N 33/5011; A61K 2035/11; G01N 2333/315; G01N 2500/10; G01N 2333/245

(57) ABSTRACT

Provided are methods for determining the health status of subject considered as a potential intestinal microflora donor, and to compositions including bacteria obtained from healthy subjects. Further provided are methods of identifying individuals of exceptional health suitable to be donors of bacteria of the intestinal microflora, and methods for producing reproducibly effective probiotic compositions, and probiotic compositions derived from such bacteria.

### FIELD OF THE INVENTION

The present invention relates to methods of identifying individuals of exceptional health, suitable to be donors of bacteria of the intestinal microflora for producing reproducibly effective pharmaceutical compositions, and to pharmaceutical compositions derived from such bacteria.

### BACKGROUND OF THE INVENTION

The intestinal microflora is a very complicated ecosystem. Any individual has at least 17 families of bacteria, 50 genera and 400-500 species and an indefinite number of subspecies. Intestinal microflora is divided into obligate microflora, i.e. microorganisms that are a constant part of the normal flora and play a role in the metabolism and anti-infective protection, and optional microflora, i.e. microorganisms commonly found in healthy people, but are opportunistic, i.e. capable of causing disease while reducing the number of non-pathological microorganisms. Dominating the obligate microflora are the anaerobic bacteria bifidobacteria and lactobacilli, which constitute about 98% by weight of gut bacteria.

Microbial populations found on or inside the body are normally benign or beneficial. These beneficial and appropriately sized microbial populations carry out a variety of helpful and necessary functions, such as aiding in digestion. They also protect the body from the penetration of pathogenic microbes. These beneficial microbial populations compete with each other for space and resources and outnumber human cells by a factor of about 10:1.

Dysbiosis (also called dysbacteriosis) refers to microbial imbalance on or inside the body. Dysbiosis is most commonly reported as a condition in the digestive tract. It has been associated with illnesses, such as inflammatory bowel disease, chronic fatigue syndrome, obesity, cancer and colitis. The term “dysbiosis” is not a standard medical term. Similar concepts are also described as “microbial imbalance”, “bacterial imbalance”, or “increased levels of harmful bacteria and reduced levels of the beneficial bacteria”. One of the ways to treat bacterial dysbiosis is the replacement of the intestinal microflora. To do this, one can either use bacterial products (probiotics) or fresh feces taken from a healthy person (fecal microbiota transplantation—FMT). In both cases, bacteria need to enter into the intestine of patients. In the case of probiotics, usually a powder of lyophilized bacteria is ingested. In the case of FMT, an emulsion of feces in water is administered into the stomach of the patient through a tube, or into the intestines through enema.

The term “probiotic” is usually used to name ingested microorganisms associated with beneficial effects to humans and other animals A significant expansion of the potential market for probiotics has led to higher requirements for scientific substantiation of putative beneficial effects conferred by the microorganisms. The World Health Organization's 2001 definition of probiotics is “live micro-organisms which, when administered in adequate amounts, confer a health benefit on the host”. This definition, although widely adopted, is not acceptable to the European Food Safety Authority because it embeds a health claim which is not measurable. A consensus definition of the term “probiotics”, based on the available information and scientific evidence, was adopted after a joint Food and Agricultural Organization of the United Nations and World Health Organization expert consultation. In October 2001, this expert consultation defined probiotics as “live micro-organisms which, when administered in adequate amounts, confer a health benefit on the host”.

Probiotics are under considerable research, as the concept holds promise for human health and well-being, and corresponding commercial opportunities. Protection of consumers requires health claims to be confirmed with sufficient scientific evidence. Overall scientific demonstration of probiotic effects requires defining a healthy microbiota and interactions between microbiota and host, and the difficulty to characterize probiotic effectiveness in health and disease. Probiotics of bacteria taken from healthy individuals are beneficial to human health in the treatment of various diseases of the gastrointestinal tract, generally known as dysbiosis, as defined above. However, there are conflicting data in the literature about the presence or absence of any therapeutic effect of probiotics. This inconsistency is primarily due to the fact that there are no criteria to identify or select certain individuals to act as donors of probiotic bacteria, except generally relying on the individuals' health and lack of apparent diseases. Therefore, individuals who were used as donors were occasionally not completely healthy. Bacteria taken from unhealthy people cannot give a positive therapeutic effect, and may indeed deteriorate their health.

It is commonly accepted that potential donors of probiotic bacteria should be screened to verify their health. For example, Stephen M. Vindigni and coworkers have recently suggested (Expert Review of Gastroenterology & Hepatology, 2013, Vol. 7(7), pages 615-628) that an effective stool donor can be a spouse, close relative or healthy unrelated donor. The probiotic therapy research center of Australia suggests that donors can be selected from individual's family members or can be close friends and disclose that all donors are fully screened for infections (parasitic, bacterial and viral) before therapy for HIV; Hep A, B, C; CMV; EBV; toxoplasmosis; syphilis; as well as for stool pathogens. Based on accumulated experience with probiotic products of varied therapeutic efficacies, such criteria can hardly be considered sufficient.

U.S. Pat. No. 5,344,762 discloses a method for early diagnosis of human cancer, wherein a human fecal sample of bacteria (Escherichia coli and/or Streptococcus faecalis) is incubated in vitro with a standard culture of a known number of cancer cells, for a period of time sufficient to enable the extent of interaction between the bacteria and the standard culture of cancer cells to be determined. The number of the interacted and/or non-interacted cancer cells present at the end of the period is determined and is utilized for the diagnosis based on the calculation of a tumor cell necrosis index (TCNI). The extent of interaction referred to may be calibrated against analogous interaction using a control preparation of bacteria. U.S. Pat. No. 5,344,762 also discloses the following four strains, isolated from human feces, which have been deposited with the American Type Culture Collection (A.T.C.C.) under the Budapest Treaty.

U.S. Pat. No. 7,449,340 discloses a method for diagnosis of malignant neoplasms derived from epithelial tissue cells in a subject, which comprises obtaining at least a first and second fecal samples from the subject, treating the fecal samples to obtain feces-derived bacteria samples, identifying one or more types of bacteria in the feces-derived bacteria samples, determining for each of the one or more types of bacteria its relative fraction from a total count of bacteria in one of the feces-derived bacteria samples, isolating one or more types of bacteria from one or both of the feces-derived bacteria samples, preparing a diagnostic sample containing bacteria of the one or more types isolated, the fraction of each of the one or more types of bacteria in the diagnostic sample corresponding to the relative fraction thereof in the fecal samples, interacting the diagnostic sample with cells for a time period sufficient to detect lysis of the cells, thereby determining for the fecal sample a TCNI, and diagnosing the subject as having or not having a malignant neoplasms derived from epithelial tissue cells in accordance with the TCNI value determined.

There remains an unmet need in the field of probiotics for a reliable method and criteria to identify the health status of potential donors of intestinal microflora, so that probiotic products having reproducible therapeutic effects may be developed.

### SUMMARY OF THE INVENTION

The present invention relates to methods and criteria for determining the suitability of a subject to provide intestinal microflora for the preparation of probiotic products. More specifically, the present invention provides methods to determine the suitability of a subject to provide intestinal microflora based on the oncolytic activity of his intestinal microflora. According to these methods, only subjects the intestinal microflora of which is determined to be highly cytotoxic and/or oncolytic are found suitable to be intestinal microflora donors for the production of probiotic products. The present invention further relates to compositions and mixtures of intestinal micro-flora obtained or derived from the intestinal microflora of healthy human donors.

The present inventions stems from several unexpected findings, which when utilized together, provide reliable determination of the level of health of potential donors of bacteria. More specifically, it has been surprisingly found that the improved methods and novel criteria provided by the present invention are able to unequivocally distinguish between healthy subjects and cancer patients based on the level of the oncolytic activity of their intestinal bacteria.

One of the main advantages of the methods provided by the present invention over methods known in the art is their superior accuracy in determining the oncolytic activity of aerobic bacteria. This improved accuracy is achieved by minimal manipulation of the cells in the assay system to decrease any induction of artifacts due to stress to the assay cells. First, after attachment to the surface of the culture vessel in which they will be contacted with the test bacteria sample or the control aerobic bacteria sample, the cancer cells are not moved or transferred throughout the assay. Thus, no enzymatic removal or resuspension step of the cells is required, since their viability is determined in the same culture vessel in which they are contacted with the aerobic bacteria samples. In addition, no centrifugation step is required. These factors, among others, substantially eliminate physical stress to the cancer cells throughout the method. As the entire method is performed in the same culture vessel, and since the cancer cells are adherent to the culture vessel, there is no need in chemically or otherwise detaching the cancer cells from the culture vessel walls. This factor, among others, substantially eliminates any kind of chemical stress to the cancer cells throughout the method. Second, the viability of substantially all the cancer cells in the culture vessel is determined, since the number of cancer cells (dead, alive and total) may be determined by automatic, electronic digital means. This factor substantially eliminates any kind of bias in selecting certain visual fields in which the cancer cells' viability would be determined, and any kind of miscalculations, in case the visual fields selected do not reflect the true viability status of all the cancer cells in the culture vessel.

The present invention provides, in one aspect, a method for determining the health status of a potential donor of intestinal microflora, comprising the steps of (i) providing a culture vessel having adherent cancer cells under standard culture conditions; (ii) determining a control baseline oncolytic level by determining the number of viable cancer cells or the number of dead cancer cells in the culture vessel of (i); (iii) inoculating a culture vessel having adherent cancer cells under standard culture conditions with a test bacterial sample comprising intestinal aerobic bacteria derived from the potential donor; (iv) incubating the inoculated culture vessel of (iii) under conditions sufficient to enable lysis of the adherent cancer cells by the intestinal aerobic bacteria; (v) determining a test oncolytic level by determining the number of viable cancer cells or the number of dead cancer cells in the inoculated culture vessel of step (iv); (vi) determining for the test bacterial sample a tumor cell necrosis index (TCNI) with the equation (A−B)/A×100=C, wherein C is the tumor cell necrosis index (TCNI), A is the number of viable cancer cells in the culture vessel of step (i) without incubation with a control oncolytic bacteria as determined in step (ii), or the number of dead cancer cells in the culture vessel of step (i) after incubation with a control oncolytic bacteria as determined in step (ii); B is the number of viable cancer cells as determined in step (v); and (vii) determining the health status of said subject in accordance with the TCNI value determined in step (vi) wherein a TCNI value in the range of 71 to 100 is indicative of said potential donor being sufficiently healthy to become a donor of intestinal microflora; wherein the method does not comprise removal of said adherent cancer cells from the culture vessel.

In certain embodiments, said culture vessel of step (iii) is the same culture vessel of step (i). In certain embodiments, said adherent cancer cells of step (iii) are the same adherent cancer cells of step (i).

In certain embodiments, the method further comprises inoculating the culture vessel of step (i) with a control bacterial sample comprising at least one control oncolytic bacterial strain prior to step (ii). In certain embodiments, the control bacterial sample is added to the culture vessel of step (i) up to about 3 hours prior to step (ii). In certain embodiments, the method further comprises incubating the inoculated culture vessel of step (i) under conditions sufficient to enable lysis of the adherent cancer cells by the control oncolytic bacterial strain prior to step (ii). In certain embodiments, the control oncolytic bacterial strain is selected from the group consisting of Escherichia coli, a Streptococcus, and any combination thereof. Each possibility represents a separate embodiment of the invention.

In certain embodiments, the test bacterial sample comprises at least one bacterial strain selected from the group consisting of Escherichia coli, a Streptococcus, Enterococcus faecalis, Enterococcus faecium, and any combination thereof. In certain embodiments, the bacterial strain is Escherichia coli. In certain embodiments, the control or test bacterial sample comprises a bacterial strain isolated from bacterial colonies formed on one or more selective culture mediums. Each possibility represents a separate embodiment of the invention.

In certain embodiments, the adherent cancer cells are derived from a solid tumor. In certain embodiments, the adherent cancer cells comprise human cancer cells. In certain embodiments, the standard culture conditions are about 37° C., 5% CO2 and 95% relative humidity (RH).

In certain embodiments, the control baseline oncolytic level of step (ii) and the test oncolytic level of step (v) are each independently the number of the viable cancer cells in the respective culture vessel. In certain embodiments, the control baseline oncolytic level of step (ii) and the test oncolytic level of step (v) are each independently the number of dead cancer cells in the respective culture vessel. In certain embodiments, the control baseline oncolytic level of step (ii) is the number of viable cancer cells in the respective culture vessel, and the test oncolytic level of step (v) is the number of dead cancer cells in the respective culture vessel. In certain embodiments, the control baseline oncolytic level of step (ii) is the number of dead cancer cells in the respective culture vessel, and the test oncolytic level of step (v) is the number of viable cancer cells in the respective culture vessel.

In certain embodiments, the duration of the incubation is about 2 to about 8 hours. In certain embodiments, the duration of the incubation is about 280 to about 400 minutes.

In certain embodiments, the determination is performed via automated means. In certain embodiments, the automated means are configured to detect a signal correlative to the number of viable and/or dead cancer cells in said culture vessel of step (ii) or step (v). In certain embodiments, the automated means are selected from the group consisting of a camera, a microscope, a photometer, a spectrophotometer, a fluorometer, and any combination thereof. In certain embodiments, the automated means are configured to analyze a signal correlative to the number of viable and/or dead cancer cells in said culture vessel of (ii) or (v). In certain embodiments, the automated means are selected from the group consisting of a computer, a signal-analyzing software, and any combination thereof. Each possibility represents a separate embodiment of the invention.

In certain embodiments, the conditions sufficient to enable lysis of the adherent cancer cells are calibrated by the extent of interaction and/or between a control oncolytic bacteria and a control culture of adherent cancer cells. In certain embodiments, the control oncolytic bacteria comprises at least one oncolytic bacterial strain selected from the group consisting of Escherichia coli, a Streptococcus, and any combination thereof. Each possibility represents a separate embodiment of the invention.

In certain embodiments, the method does not comprise a step inflicting a chemical or physical insult to the cancer cells of said culture vessel. In certain embodiments, the step inflicting a chemical or physical insult is selected from the group consisting of use of trypsin, use of a cell scraper, formation of a cell suspension, use of a toxic dye, centrifugation, and any combination thereof. Each possibility represents a separate embodiment of the invention.

In certain embodiments, the ratio between the number of bacteria in the test bacterial sample or in the control bacterial sample and the number of adherent cancer cells in the respective culture vessel is about 10⁶ to about 10⁷. In certain embodiments, the test bacterial sample or the control bacterial sample comprises about 10⁹ to 10¹¹ of their respective bacteria.

In certain embodiments, the number of viable and/or dead cancer cells in said culture vessel of (i) is determined (a) in the presence of a control oncolytic bacterial strain after co-incubation under conditions sufficient to enable lysis of the cancer cells by the control oncolytic bacterial strain, (b) after the addition of a control oncolytic bacterial strain but before the control oncolytic bacterial strain is able to lyse the adherent cancer cells, (c) after the addition of a control non-oncolytic bacterial strain, or (d) without the addition of any bacteria. Each possibility represents a separate embodiment of the invention.

In certain embodiments, a TCNI value in the range of 76 to 100, 81 to 100, 90 to 100 or 95 to 100, or a TCNI value of 100 is indicative of the subject being sufficiently healthy to become a donor of intestinal microflora. In certain embodiments, a TCNI value in the range of 71 to 85, 74 to 85, 71 to 81 or 76 to 80 is indicative of the subject being sufficiently healthy to become a donor of intestinal microflora. Each possibility represents a separate embodiment of the invention.

The present invention further provides, in another aspect, a composition comprising at least one aerobic oncolytic bacteria, wherein the at least one aerobic oncolytic bacteria is obtained, isolated or derived from a healthy donor, wherein the healthy donor is tested to have a TCNI value in the range of 71 to 100 by any one of the methods described above.

In certain embodiments, the at least one aerobic oncolytic bacteria is selected from the group consisting of Escherichia coli, a Streptococcus, Enterococcus faecalis, Enterococcus faecium, and any combination thereof. In certain embodiments, the bacterial strain is Escherichia coli. In certain embodiments, the at least one aerobic oncolytic bacteria is selected from the group consisting of Escherichia coli A.T.C.C. 55373, Escherichia coli A.T.C.C. 55374, Escherichia coli A.T.C.C. 55375, Streptococcus faecalis A.T.C.C. 55376, and any combination thereof. In certain embodiments, the composition comprises Escherichia coli A.T.C.C. 55373, Escherichia coli A.T.C.C. 55374, Escherichia coli A.T.C.C. 55375, and Streptococcus faecalis A.T.C.C. 55376. Each possibility represents a separate embodiment of the invention.

In certain embodiments, the composition is tested to have a TCNI value in the range of 71 to 100. In certain embodiments, the at least one aerobic oncolytic bacteria is tested to have a TCNI value in the range of 71 to 100. In certain embodiments, the composition further comprises at least one additional strain of bacteria, the composition thus comprising a mixture of bacteria, wherein the mixture is tested to have a TCNI value in the range of 71 to 100.

In certain embodiments, the at least one additional strain of bacteria is anaerobic bacteria. In certain embodiments, the anaerobic bacteria are selected from an anaerobic Bifidobacterium species, an anaerobic Lactobacillus species, and any combination thereof. In certain embodiments, the at least one additional strain of bacteria is selected from the group consisting of Enterococcus faecalis and Enterococcus faecium. Each possibility represents a separate embodiment of the invention.

In certain embodiments, the anaerobic Bifidobacterium species is selected from the group consisting of Bifidobacterium breve, Bifidobacterium longum, and Bifidobacterium infantis. Each possibility represents a separate embodiment of the invention. In certain embodiments, the anaerobic Bifidobacterium species comprise a mixture of Bifidobacterium breve, Bifidobacterium longum, and Bifidobacterium infantis. Each possibility represents a separate embodiment of the invention. In certain embodiments, the anaerobic Lactobacillus species is selected from the group consisting of Lactobacillus acidophilus, Lactobacillus plantarum, Lactobacillus paracasei, and Lactobacillus delbrueckii subsp. Bulgaricus. Each possibility represents a separate embodiment of the invention. In certain embodiments, the anaerobic Lactobacillus species comprise a mixture of Lactobacillus acidophilus, Lactobacillus plantarum, Lactobacillus paracasei, and Lactobacillus delbrueckii subsp. Bulgaricus. In certain embodiments, the anaerobic Streptococcus species is Streptococcus thermophiles. In certain embodiments, the anaerobic Bifidobacterium species comprise a mixture of Bifidobacterium breve, Bifidobacterium longum, and Bifidobacterium infantis; wherein the anaerobic Lactobacillus species comprise a mixture of Lactobacillus acidophilus, Lactobacillus plantarum, Lactobacillus paracasei, and Lactobacillus delbrueckii subsp. Bulgaricus; and wherein the anaerobic Streptococcus species is Streptococcus thermophiles.

In certain embodiments, the composition further comprises nutritional supplements for aerobic bacteria, selected from the groups consisting of peptides, peptones, vitamins, trace elements, minerals, and any combination thereof. In certain embodiments, the nutritional supplements are selected from the groups consisting of tryptone, yeast extract, sodium chloride, glucose, and any combination thereof. Each possibility represents a separate embodiment of the invention.

As described above, besides aerobic highly-oncolytic Escherichia coli (95-100% TCNI) and Streptococcus faecalis, many aerobic partial-oncolytic (50-94% TCNI) or non-oncolytic (0-49% TCNI) bacteria species are known and may be added to the composition. In certain embodiments, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% by weight of all aerobic bacteria in the composition are aerobic highly-oncolytic bacteria. Each possibility represents a separate embodiment of the invention. In certain embodiments, all of the aerobic bacteria in the composition are aerobic highly-oncolytic bacteria. In certain embodiments, the aerobic bacteria in the composition have a mean TCNI value of at least 50, at least 60, at least 70, at least 80, at least 90, at least 95, at least 96, at least 97, at least 98, at least 99, or 100. Each possibility represents a separate embodiment of the invention.

In certain embodiments, the TCNI value is in the range of 76 to 100, 81 to 100, 90 to 100, or 95 to 100, or the TCNI value is 100.

The present invention further provides, in another aspect, an oral dosage form, comprising any one of the compositions described above.

The present invention further provides, in another aspect, a rectal dosage form, comprising any one of the compositions described above.

The present invention further provides, in another aspect, a pharmaceutical composition comprising any one of the compositions described above.

The present invention further provides, in another aspect, any one of the compositions described above for use in treating a dysbiosis-related disease or condition.

In certain embodiments, the dysbiosis-related disease or condition is selected from the group consisting of dysbiosis, cancer, inflammatory bowel disease, chronic fatigue syndrome, obesity and colitis. Each possibility represents a separate embodiment of the invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a schematic illustration of an embodiment of the method provided by the present invention, illustrating aerobic bacteria extraction from a subject's digestive system waste product.

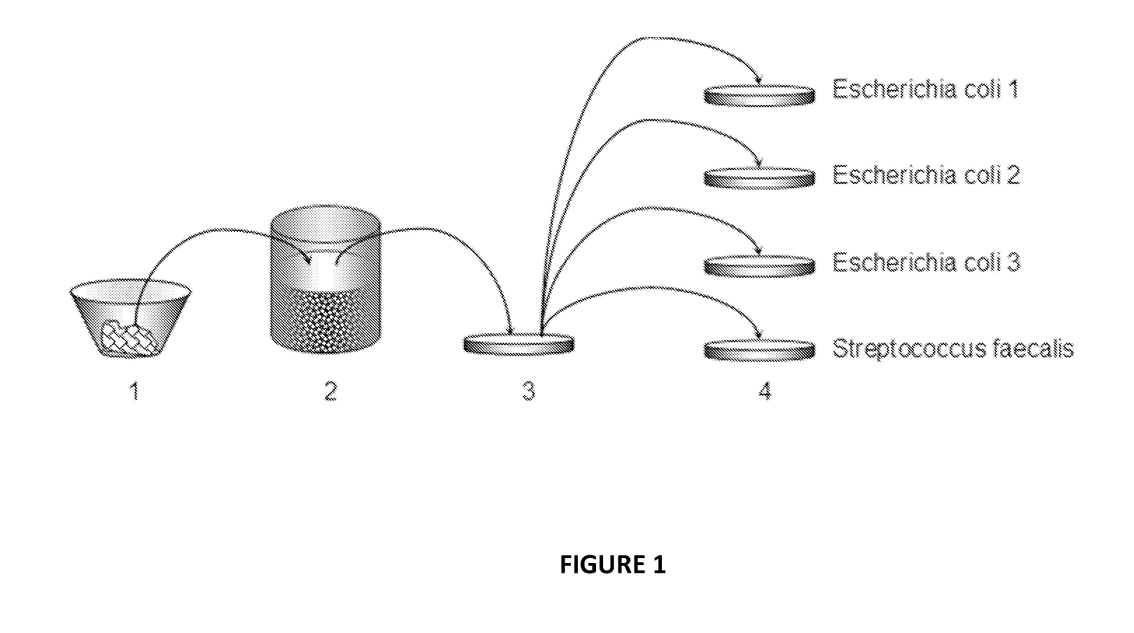

FIG. 2 is a schematic illustration of an embodiment of the method provided by the present invention, illustrating a direct method for determining the number of live and/or dead cancer cells in the culture vessel intended for contact with aerobic bacteria.

FIG. 3 is a schematic illustration of an embodiment of the method provided by the present invention, illustrating an indirect method for determining the number of live and/or dead cancer cells in the culture vessel intended for contact with aerobic bacteria.

FIG. 4 illustrates the results of a clinical trial determining the tumor cell necrosis index (TCNI) for populations of healthy subjects and cancer patients.

### DETAILED DESCRIPTION OF THE INVENTION

The present invention provides methods, as well as guidelines and selection criteria for determining the suitability of a subject to provide intestinal microflora for the preparation of probiotic products. More specifically, the present invention provides methods to determine the suitability of a subject to provide intestinal microflora based on the oncolytic activity of his intestinal microflora. According to these methods, only subjects the intestinal microflora of which is determined to be highly oncolytic are found suitable to be donors of intestinal microflora for the production of probiotic products.

The advantages of the methods provided by the present invention over other methods are several folds. For example, accuracy is improved, thus providing selection criteria which practically eliminate the occurrence of diseased patients mistakenly identified as healthy subjects. In addition, the methods provided herein require steps which do not necessitate human intervention, making the methods robust, high-throughput, and more adequate for commercial use.

Using the method described in U.S. Pat. No. 5,344,762, the author has found that healthy subjects had a TCNI of 68-100 (and an average TCNI of 86), patients having various classes of diseases had a TCNI of 21-100, and pre-operative oncological patients had a TCNI of 10-49 (with an average TCNI of 29). According to the methods provided in U.S. Pat. No. 7,449,340, using a TCNI value of 70 as cutoff only reached 61% sensitivity for normal, non-cancer subjects and 74% sensitivity for subjects with active cancer. The methods disclosed in the present invention are superior to those disclosed, for example, in U.S. Pat. No. 5,344,762 and U.S. Pat. No. 7,449,340.

The prior art methods disclosed therein suffer from two inherent major drawbacks when brought into practice. First, these methods require active physical and/or chemical manipulation of the cancer cells utilized to test the patient-derived bacteria sample. For example, after making the suspension of cancer cells, the cells are transferred to a glass slide for drying and staining, and thus part of live cells may appear as dead cells due to their punctured membrane. These manipulations damage the cancer cells employed by the methods to test the bacterial oncolytic capability, thereby severely hampering the accuracy of these methods, thus risking poor or miss-diagnosis of potential cancer patients. In other words, these methods are prone to false-negative results, i.e. when a cancer patient is diagnosed to be healthy, and thus does not receive appropriate treatment. Second, these methods require considerable amounts of manual labor. As a result, a single person, such as a lab technician, can perform not more than 5-10 assays a day, while routinely required to perform hundreds or more of these tests a day.

As a result of these drawbacks, in many cases there can be either hyper- or hypo-diagnosis (false-positive or false-negative, respectively), which may be life-threatening for cancer patients if not diagnosed on time and/or result in unnecessarily and profoundly damaging the quality of life of misdiagnosed subjects.

The present invention provides methods of measurement of oncolytic activity of aerobic human intestines aerobic flora, which are free from the above mentioned technical shortcomings. These methods exclude any steps which may cause any chemical and/or mechanical shock to the tested cancer cells. As a result, the accuracy of measurements of oncolytic activity of aerobic human intestines aerobic flora increases and therefore miss-identification of a diseased subject as healthy is avoided or at least substantially minimized

The present invention thus provides, in one aspect, a method for determining the health status of a potential donor of intestinal microflora, comprising the steps of (i) providing a culture vessel having adherent cancer cells under standard culture conditions; (ii) determining a control baseline oncolytic level by determining the number of viable cancer cells or the number of dead cancer cells in the culture vessel of (i); (iii) inoculating a culture vessel having adherent cancer cells under standard culture conditions with a test bacterial sample comprising intestinal aerobic bacteria derived from the potential donor; (iv) incubating the inoculated culture vessel of (iii) under conditions sufficient to enable lysis of the adherent cancer cells by the intestinal aerobic bacteria; (v) determining a test oncolytic level by determining the number of viable cancer cells or the number of dead cancer cells in the inoculated culture vessel of step (iv); (vi) determining for the test bacterial sample a tumor cell necrosis index (TCNI) with the equation (A−B)/A×100=C, wherein C is the tumor cell necrosis index (TCNI), A is the number of viable cancer cells in the culture vessel of step (i) without incubation with a control oncolytic bacteria as determined in step (ii), or the number of dead cancer cells in the culture vessel of step (i) after incubation with a control oncolytic bacteria as determined in step (ii); B is the number of viable cancer cells as determined in step (v); and (vii) determining the health status of said subject in accordance with the TCNI value determined in step (vi) wherein a TCNI value in the range of 71 to 100 is indicative of said potential donor being sufficiently healthy to become a donor of intestinal microflora; wherein the method does not comprise removal of said adherent cancer cells from the culture vessel.

The term “intestinal microflora” or “intestinal aerobic bacteria” as used herein refers to any aerobic bacteria found in, obtained, derived or isolated by any technique from the digestive tract of a human. The term “aerobic bacteria” as used herein refers to any bacteria which are obligate aerobes, i.e. which need oxygen to grow, facultative anaerobes, i.e. which use oxygen if it is available, but also have anaerobic methods of energy production, microaerophiles, i.e. which require oxygen for energy production, but are harmed by atmospheric concentrations of oxygen (21% 02), or aero-tolerant anaerobes, i.e. which do not use oxygen but are not harmed by it.

The terms “donor”, “potential donor”, “subject”, “individual”, “patient” or “mammal” as are used herein, mean any subject, particularly a mammalian subject, for whom any test, screening, diagnosis or therapy is desired. Mammalian subjects include humans, domestic animals, farm animals, and zoo, sports, or pet animals such as dogs, cats, guinea pigs, rabbits, rats, mice, horses, cattle, cows, and so on.

The term “culture vessel” is used herein in its broadest sense, and used as synonym for any kind of a container suitable for the tests, screening, experiments and methods described or provided by the present invention. The term “screening” as used herein refers to the capability of the methods provided herein to be performed or executed in short periods of time, in small volumes, and/or by automated means, such that at least 6, at least 12, at least 24, at least 48, at least 96, at least 384 or more tests can be done simultaneously in a single culture vessel. Each possibility represents a separate embodiment of the invention. In certain embodiments, a plurality of control or and/or test bacterial samples are tested in standardized cultures of adherent cancer cells in a single culture vessel. Each possibility represents a separate embodiment of the present invention. In certain embodiments, the culture vessel comprises a flat horizontal bottom. In certain embodiments, the culture vessel comprises a U-shape bottom. A non-limiting example of a culture vessel comprising a flat horizontal bottom is multi-well plate, a flask or a petri dish. In certain other embodiments, the culture vessel has the shape of a cylinder. A non-limiting example of a culture vessel which has the shape of a cylinder is a roller bottle or a test tube. In certain embodiments, a plurality of control or and/or test bacterial samples are tested in standardized cultures of adherent cancer cells in a single culture vessel. In certain embodiments, said culture vessel is selected from the group consisting of a 2-well plate, a 4-well plate, a 6-well plate, a 12-well plate, a 24-well plate, a 48-well plate, a 96-well plate, and a 384-well plate. Each possibility represents a separate embodiment of the present invention.

In certain embodiments, the culture vessel comprises a predetermined number of cancer cells, i.e. the number of cell is determined before the cells were added to the culture. In certain embodiments, the culture vessel comprises a determined number of cancer cells, i.e. the number of cell is determined after the cells were added to the culture. In certain embodiments, the culture vessel comprises live cancer cells, dead cancer cells, and any combination thereof. In certain embodiments, the culture vessel prior to any incubation with bacteria comprises more than 80%, more than 85%, more than 90%, more than 95% or more than 99% living cancer cells. Each possibility represents a separate embodiment of the invention. In certain embodiments, the culture vessel comprises less than 20%, less than 15%, less than 10%, less than 5% or less than 1% dead cancer cells prior to any incubation with bacteria. Each possibility represents a separate embodiment of the invention. In certain embodiments, the adherent cancer cells in the culture vessel are at least 50%, at least 60%, at least 70%, at least 80%, at least 85%, at least 90%, at least 95% or at least 99% confluent prior to any incubation with bacteria. In certain embodiments, the adherent cancer cells in the culture vessel are 60-80% confluent. Each possibility represents a separate embodiment of the invention. In certain embodiments, the cancer cells of the standardized culture are in monolayer.

The phrase “adherent cancer cells under standard culture conditions” as used herein refers to a culture of cancer cells which adhere to the culture vessel, kept under appropriate conditions to allow the cells to live without significant stress. For example, cancer cell lines and cultures of primary cells (i.e. non-replicating cells) are routinely grown and passaged in a sterile environment, at 37° C., 5% CO₂ and 95% relative humidity. An exemplary cell line of adherent cancer cells suitable for the methods provided herein is HCT 116 (colorectal carcinoma; ATCC CCL-24) and/or MCF7 (adenocarcinoma; ATCC HTB-2).

The term “control baseline oncolytic level” as used herein refers to the number of viable cancer cells, the number of dead cancer cells, or the total number of cancer cells, in a culture vessel, measured in the presence or absence of bacteria. In certain embodiments, the control baseline oncolytic level is the number of viable adherent cancer cells in the culture vessel before the addition of any bacteria. In certain embodiments, the control baseline oncolytic level is the number of viable adherent cancer cells in the culture vessel after the addition of a control non-oncolytic bacterial strain. In certain embodiments, the control baseline oncolytic level is the number of viable adherent cancer cells in the culture vessel after the addition of a control oncolytic bacterial strain but before the control oncolytic bacterial strain is able to lyse the adherent cancer cells, or (iv) the number of cells in the culture vessel lysed by an oncolytic control aerobic bacteria after incubation under conditions sufficient to enable lysis.

The phrase “determining the number of viable cancer cells” as used herein refers to the use of any method known in the art for directly or indirectly identify the presence of a live cell or a plurality of live cells, e.g. cancer cells, and obtaining a corresponding number. The phrase “determining the number of dead cancer cells” as used herein refers to the use of any method known in the art for directly or indirectly identify the presence of a dead cell or a plurality of dead cells, e.g. cancer cells, and obtaining a corresponding number. The term “viable” is interchangeable with the term “live”. The terms “viable cancer cell” and “dead cancer cell” as used herein refer to any cancer cell or a plurality of cancer cells determined or identified by any one of numerous methods for determination of cell viability known in the field to be alive or dead, respectively. For example, Trypan blue is a stain used to selectively color dead tissues or cells blue. Therefore, any cell dyed blue during Trypan blue staining is considered and counted as a “dead cancer cell”, and vice versa, any cell not dyed blue during Trypan blue staining is considered and counted as a “viable cancer cell”. The phrase “determining the total number of cancer cells” as used herein refers to the use of any method known in the art for identifying the presence of any cell or a plurality of cells or a population of cells, e.g. cancer cells, and obtaining a corresponding number.

It should be emphasized that the phrases “determining the number of viable cancer cells” and “determining the number of dead cancer cells” as used herein further refer to determining the level or value of any signal which is exclusively emitted by viable or dead cancer cells, respectively, either directly or indirectly. It should be further emphasized that the terms “control baseline oncolytic level” and “test oncolytic level” as used herein further refer to a level or value of any signal which is exclusively emitted by viable or dead cancer cells, either directly or indirectly. It should also be emphasized that the terms “A” and “B” while calculating a TCNI value further refer to a level or value of any signal which is exclusively emitted by viable or dead cancer cells, either directly or indirectly.

In certain embodiments, determining the number of viable cancer cells is performed on cancer cells which are viable. In certain embodiments, determining the number of viable cancer cells is performed on cancer cells which are viable and continue to stay viable during the method. In certain embodiments, determining the number of viable cancer cells is performed on cancer cells which are not subjected to any cytotoxic preparation step in order to determine their number. In certain embodiments, the cytotoxic preparation step is fixation, staining, dehydration or any combination thereof. In certain embodiments, the cytotoxic preparation step is fixation.

In certain embodiments, the number of dead cancer cells and the number of viable cancer cells are determined and summed, thereby indirectly determining the total number of cancer cells. In certain embodiments, the total number of cancer cells is determined, thereby directly determining the total number of cancer cells. In some embodiments, the number of dead cancer cells is determined directly. In some embodiments, the number of dead cancer cells is determined indirectly by subtraction of the number of viable cancer cells from the total number of cancer cells. In some embodiments, the number of viable cancer cells is determined directly. In some embodiments, the number of viable cancer cells is determined indirectly by subtraction of the number of dead cancer cells from the total number of cancer cells. In some embodiments, the number of dead and/or viable cancer cells is determined by measuring the concentration of a substance not existing initially in the solution and which appeared from the protoplasm of dead cancer cells as a result of cell integrity destruction. In some embodiments, the substance is selected from the group consisting of a protein, an enzyme, a lipid, a sugar, an organelle, and any combination thereof. Each possibility represents a different embodiment of the invention. In some embodiments, the number of dead and/or viable cancer cells is determined by a combination of direct and indirect methods.

The term “inoculating” as used herein generally refers to the addition of a control bacterial sample or a test bacterial sample to a culture vessel.

The term “test bacterial sample” as used herein refers to any sample comprising or consisting of at least one aerobic bacterial strain obtained or isolated, either directly or indirectly, from a human intestine. Examples of test bacterial sample comprise, but are not limited to, a fecal sample obtained directly from the intestine of a patient without further processing, a fecal sample obtained from a feces sample of a patient without further processing, a fecal sample obtained from a feces sample emulsified in a liquid such as saline or buffer, or a fecal sample decontaminated from one or more non-bacterial components.

The terms “stool sample”, “fecal sample” and “feces sample” as used herein may be used interchangeably and refer to the waste product of the human digestive system, or to any bacteria therefrom.

The terms “derived from”, “isolated from” and “obtained from” as used herein interchangeably generally refer to the source of bacteria.

The term “conditions sufficient to enable lysis” refers to the chemical and/or physical environment, and to conditions under which an oncolytic bacterium would be able to lyse a cancer cell. As many bacteria strains and many cancer cell lines are known, the specific conditions needed to enable lysis are adjusted using standardized methods known in the art. For example, it is known that the duration of incubation in order to detect lysis is dependent, at least in part, on the type of cancer cells used. The duration of incubation and the temperature of incubation have major impact on the measured oncolytic activity of the tested bacterial sample. However, other variables may have significant influence on the level of oncolytic activity being measured. Therefore, incubation conditions may be tailored to accommodate cell-bacteria interactions using methods well known in the field.

The term “oncolytic activity” as used herein refers to cytotoxic and/or morphological effect(s) exerted in-vitro and/or in-vivo on cancer cells by oncolytic bacteria. In certain embodiments, the term “oncolytic activity” means breakage or rupture of the membrane of the cancer cell. In-vitro, these effects are routinely detected by various means as known in prior art, for example, by staining with a selective stain for dead cells, by inhibition of DNA synthesis, or by apoptosis. Detection of these effects in-vivo is also performed by methods known in the art.

The term “test oncolytic level” as used herein refers to the number of viable cancer cells, to the number of dead cancer cells, or to the total number of cancer cells, in a culture vessel, measured in the presence of test bacteria, the oncolytic activity of which is been determined. In certain embodiments, the test oncolytic level is the number of viable cancer cells in a culture vessel after the addition of test bacteria and co-incubation under conditions sufficient to enable lysis.

The term “control bacterial sample” as used herein refers to any sample comprising or consisting of at least one control aerobic bacterial strain. The term “control aerobic bacterial strain” as used herein refers to any bacterial strain known or tested to be either control oncolytic bacterial strain or control non-oncolytic bacterial strain. The term “control oncolytic bacterial strain” as used herein refers to any aerobic bacterial strain known or tested to be highly cytotoxic to cancer cells. The term “control non-oncolytic bacterial strain” as used herein refers to any aerobic bacterial strain known or tested to have no or negligible influence on the viability of cancer cells.

The present invention defines the term “tumor cell necrosis index” or “TCNI” with the following formula:

$$T C N I ( \% ) = \frac{100 ( A - B )}{A}$$

in which “A” is (i) the number of viable adherent cancer cells in the culture vessel before the addition of any bacteria, (ii) the number of viable adherent cancer cells in the culture vessel after the addition of a control non-oncolytic bacterial strain, (iii) the number of viable adherent cancer cells in the culture vessel after the addition of a control oncolytic bacterial strain but before the control oncolytic bacterial strain is able to lyse the adherent cancer cells, or (iv) the number of cells in the culture vessel lysed by an oncolytic control aerobic bacteria after incubation under conditions sufficient to enable lysis. In all cases, “A” would be a relatively high number, as the number of viable adherent cancer cells in the culture vessel in the beginning of the method (before lysis), and the number of dead cancer cells in the culture vessel after incubation with an oncolytic control aerobic bacteria (after lysis) is expected to be high. “B” is the number of viable adherent cancer cells in the culture vessel after the addition of test bacteria. In practice, when the test bacteria are taken from healthy subjects having oncolytic intestinal bacteria, “B” will be relatively low, giving a relatively high TCNI (up to 100%). In other cases, when the test bacteria are taken from cancer patients having less oncolytic or non-oncolytic intestinal bacteria, “B” will be relatively high, giving a relatively low TCNI (down to 0%).

In certain embodiments, “A” is the number of viable adherent cancer cells in the culture vessel before the addition of any bacteria. In certain embodiments, “A” is the number of viable adherent cancer cells in the culture vessel after the addition of a control non-oncolytic bacterial strain. In certain embodiments, “A” is the number of viable adherent cancer cells in the culture vessel after the addition of a control oncolytic bacterial strain but before the control oncolytic bacterial strain is able to lyse the adherent cancer cells. In certain embodiments, “A” is the number of cells in the culture vessel lysed by an oncolytic control aerobic bacteria after incubation under conditions sufficient to enable lysis. In certain embodiments, “B” is the number of viable adherent cancer cells in the culture vessel after the addition of test bacteria and after co-incubation under conditions sufficient to enable lysis.

The phrase “sufficiently healthy to become a donor of intestinal microflora” as used herein refers to a subject having oncolytic intestinal microflora. In certain embodiments, a donor of intestinal microflora is a subject not afflicted by any disease or condition known or tested to induce or aggravate dysbiosis. In certain embodiments, a donor of intestinal microflora is not afflicted by dysbiosis, cancer, inflammatory bowel disease, chronic fatigue syndrome, obesity and colitis. Each possibility represents a separate embodiment of the invention. In certain embodiments, a donor of intestinal microflora is not afflicted by dysbiosis. In certain embodiments, a donor of intestinal microflora is not afflicted by cancer.

In certain embodiments, the culture vessel inoculated with the test bacterial sample is the culture vessel of step (i). In certain embodiments, the adherent cancer cells inoculated with the test bacterial sample are the adherent cancer cells of step (i). It should be emphasized that the determination of the control baseline oncolytic level may be performed independently and/or simultaneously with the inoculation of a culture vessel with the test bacterial sample, i.e. in different culture vessels. Alternatively, the determination of the control baseline oncolytic level may be performed prior to the inoculation of the same culture vessel with the test bacterial sample. Thus, in certain embodiments, the culture vessel inoculated with the test bacterial sample is the culture vessel of step (i). Thus, in certain embodiments, all the steps of the method are performed in the same culture vessel.

In certain embodiments, said method further comprises inoculating the culture vessel of step (i) with a control bacterial sample comprising at least one control non-oncolytic bacterial strain prior to step (ii). In certain embodiments, said method further comprises inoculating the culture vessel of step (i) with a control bacterial sample comprising at least one control oncolytic bacterial strain prior to step (ii). In certain embodiments, the control bacterial sample is added to the culture vessel of step (i) up to about 1, 2 or 3 hours prior to step (ii). In certain embodiments, the method further comprises incubating the inoculated culture vessel of step (i) under conditions sufficient to enable lysis of the adherent cancer cells by the control oncolytic bacterial strain prior to step (ii). In certain embodiments, the control oncolytic bacterial strain is selected from the group consisting of Escherichia coli, a Streptococcus, and any combination thereof. Each possibility represents a separate embodiment of the present invention.

In certain embodiments, the number of live and/or dead cancer cells in the culture vessel is determined without or prior to the addition of control bacteria. In certain embodiments, the number of live and/or dead cancer cells in the culture vessel is determined after the addition of control bacteria. In certain embodiments, the number of live and/or dead cancer cells in the culture vessel is determined after the addition of control bacteria and after a sufficient period of co-incubation under conditions sufficient to enable lysis. In certain embodiments, the control bacteria are oncolytic aerobic bacteria. In certain embodiments, the control aerobic bacteria are non-oncolytic aerobic bacteria.

Gut flora or, more appropriately, gut microbiota, consists of a complex of microorganism species that live in the digestive tracts of animals and is the largest reservoir of microorganisms mutual to humans. In approximation, the human body carries about 100 trillion microorganisms in its intestines. Of those, the methods provided by the present invention make use of aerobic bacteria. In certain embodiments, the test bacterial sample comprises at least one bacterial strain selected from the group consisting of Escherichia coli, a Streptococcus, Enterococcus faecalis, Enterococcus faecium, and any combination thereof. In certain embodiments, the bacterial strain is Escherichia coli. In certain embodiments, the control or test bacterial sample comprises a bacterial strain isolated from bacterial colonies formed on one or more selective culture mediums. Each possibility represents a separate embodiment of the invention.

In addition to the use of aerobic bacteria-type-specific petri dishes, another way to minimize potential interference with the methods provided by the present application is to decontaminate the bacteria in the test bacterial sample before the sample is inoculated to the culture vessel, i.e to remove one or more components of a stool sample suspected or known to interfere with bacterial oncolytic activity. Thus, in certain embodiments, at least one contamination or contaminating material is removed from the stool sample prior to the inoculation. Further examples of test bacterial samples are those produced by e.g. preparing a water emulsion of a fecal sample and inoculating the suspension to separate, selective, bacteria-type-specific petri dishes, as known in the field. Of these petri dishes, a single type of bacteria or a combination of different type of bacteria produce the test bacterial sample.

In certain embodiments, the adherent cancer cells are derived from a solid tumor. The term “solid tumor” as used herein refers to an abnormal mass of tissue. Solid tumors may be benign (not cancer), or malignant (cancer). Different types of solid tumors are named for the type of cells that form them. Examples of solid tumors are sarcomas, carcinomas, and lymphomas. Leukemias (cancers of the blood) generally do not form solid tumors. The term “solid tumor” as used herein further refers to metastasis originated in solid tumors.

To detect and/or quantify the oncolytic capability or activity of one or more aerobic bacterial strains, these aerobic bacterial strains are added to cancer cells and evidence of oncolysis is sought after. The cancer cells used for these tests may be of any organism, tissue or cell type, as long as they are susceptible to oncolysis by at least known oncolytic bacterial strain. As the present invention is directed, in part, to methods of diagnosis a state of disease in humans, it may be beneficial to use cancer cells or cancer cell lines derived from or obtained from a human in these methods. Therefore, in certain embodiments, the adherent cancer cells are or comprise human cancer cells.

As specified above, human cancer cell lines as well as primary cell cultures are routinely kept in humidified incubators, under predetermined and controlled CO₂ levels and temperature. However, slightly elevated temperatures may promote oncolysis. In certain embodiments, the standard culture conditions are about 37° C., about 5% CO₂ and about 95% relative humidity (RH). The term “standard culture conditions” as used herein refers to the chemical and/or physical environment, and to the terms and conditions in which the adherent cancer cells are maintained in a viable state in a culture vessel.

In certain embodiments, the control baseline oncolytic level and the test oncolytic level are each independently the number of the viable cancer cells in the respective culture vessel. In certain embodiments, the control baseline oncolytic level and the test oncolytic level are each independently the number of dead cancer cells in the respective culture vessel. In certain embodiments, the control baseline oncolytic level is the number of viable cancer cells in the respective culture vessel, and the test oncolytic level is the number of dead cancer cells in the respective culture vessel. In certain embodiments, the control baseline oncolytic level is the number of dead cancer cells in the respective culture vessel, and the test oncolytic level is the number of viable cancer cells in the respective culture vessel.

As specified above, it is known that the duration of incubation in order to detect lysis is dependent, at least partly, on the type of cancer cells used. The exact duration, or an appropriate range of duration is routinely determined by a persons of average skill in the art, according to and/or using known methods of the field. In certain embodiments, the duration of the incubation is about 2 to about 8 hours. In certain embodiments, the duration of the incubation is about 280 to about 400 minutes. In certain embodiments, the duration of the incubation is at least 240 minutes. In certain embodiments, the duration of the incubation is at least 280 minutes. In certain embodiments, the duration of the incubation is not more than 400 minutes. In certain embodiments, the duration of the incubation is 240, 280, 320, 360 or 400 minutes. In certain embodiments, the duration of the incubation is 280, 320, 360 or 400 minutes. Each possibility represents a separate embodiment of the invention.

In certain embodiments, the duration of the incubation is at least 1 hour, at least 2 hours, at least 3 hours, at least 4 hours, at least 5 hours, at least 6 hours, or at least 7 hours. In certain other embodiments, the duration of the incubation is less than 8 hours, less than 7 hours, less than 6 hours, less than 5 hours, or less than 4 hours. Each possibility represents a separate embodiment of the invention.

In order to increase the robustness of the methods provided herein, many of its steps may be performed without human intervention. In certain embodiments, the determination is performed via automated means. The term “automated means” as used herein refers to one or more hardware or software which is capable of repeating its activity at least twice without human interaction with this hardware or software during its activity. For Example, a programmable fluorometer which can be programmed to measure fluorescence from at least two wells in a 96-well plate is considered an “automated mean”. As used herein, the term “automated” means that the process is one which operates by electronic means with little or, preferably, no human intervention.

In certain embodiments, the automated means are configured to detect a signal correlative to the number of viable and/or dead cancer cells in said culture vessel of step (ii) or step (v). In certain embodiments, the automated means are selected from the group consisting of a camera, a microscope, a photometer, a spectrophotometer, a fluorometer, and any combination thereof. In certain embodiments, the automated means are configured to analyze a signal correlative to the number of viable and/or dead cancer cells in said culture vessel of (ii) or (v). In certain embodiments, the automated means are selected from the group consisting of a computer, a signal-analyzing software, and any combination thereof. Each possibility represents a separate embodiment of the invention.

The term “configured” as used herein refers to a system, apparatus, structure or software that is constructed to perform a particular task or adopt a particular configuration.

The term “configured” can be used interchangeably with other similar phrases such as “arranged and configured”, “constructed and arranged”, “adapted and configured”, “adapted”, “constructed”, “manufactured and arranged”, and the like.

In certain embodiments, the conditions sufficient to enable lysis of the adherent cancer cells are calibrated by the extent of interaction and/or lysis between a control oncolytic bacteria and a control culture of adherent cancer cells, which is susceptible to lysis by the control oncolytic bacteria. In certain embodiments, the control oncolytic bacteria comprises at least one oncolytic bacterial strain selected from the group consisting of Escherichia coli, a Streptococcus, and any combination thereof. Each possibility represents a separate embodiment of the invention.

In certain embodiments, the method does not comprise a step inflicting a chemical or physical insult to the cancer cells of said culture vessel. In certain embodiments, the step inflicting a chemical or physical insult is selected from the group consisting of use of trypsin, use of a cell scraper, formation of a cell suspension, use of a toxic dye, centrifugation, and any combination thereof. Each possibility represents a separate embodiment of the invention. In certain embodiments, the method does not comprise a step of enzymatic digestion of the cancer cells of said culture vessel.

In order to detect lysis of cancer cells by control or test bacteria samples, one of the conditions needed to be determined is the ratio of bacteria to viable adherent cancer cells at the beginning of their co-incubation. Dependent on several other technical factors, this ratio must also be determined experimentally in order to achieve the most accurate results. Therefore, in certain embodiments, the ratio between the control or test bacteria to the cancer cells is at least 100,000:1. In certain embodiments, the ratio between the control or test bacteria to the cancer cells is at least 1,000,000:1. In certain embodiments, the ratio between the number of bacteria in the test bacterial sample or in the control bacterial sample and the number of adherent cancer cells in the respective culture vessel is about 10⁶ to about 10⁷. In certain embodiments, the test bacterial sample or the control bacterial sample comprises about 10⁹ to 10¹¹ of their respective bacteria. Each possibility represents a different embodiment of the invention.

In certain embodiments, the number of viable and/or dead cancer cells in said culture vessel of (i) is determined (a) in the presence of a control oncolytic bacterial strain after co-incubation under conditions sufficient to enable lysis of the cancer cells by the control oncolytic bacterial strain, (b) after the addition of a control oncolytic bacterial strain but before the control oncolytic bacterial strain is able to lyse the adherent cancer cells, (c) after the addition of a control non-oncolytic bacterial strain, or (d) without the addition of any bacteria.

U.S. Pat. Nos. 5,344,762 and 7,449,340 both relate to methods for early diagnosis of cancer, but provide different tumor cell necrosis index (TCNI) values for diagnosing tested subjects as cancer patients. While U.S. Pat. No. 5,344,762 refers to a TCNI value of 61% and higher, obtained by the therein disclosed method, as indicative of absence of malignant tumors in the body of the subject, U.S. Pat. No. 7,449,340 discloses that the therein disclosed method, with a similar TCNI value of 50%, is 86% sensitive to non-cancer patients.

In certain embodiments, a TCNI value in the range of 76 to 100 is indicative of the subject being sufficiently healthy to become a donor of intestinal microflora. In certain embodiments, a TCNI value in the range of 81 to 100 is indicative of the subject being sufficiently healthy to become a donor of intestinal microflora. In certain embodiments, a TCNI value in the range of 90 to 100 is indicative of the subject being sufficiently healthy to become a donor of intestinal microflora. In certain embodiments, a TCNI value in the range of 95 to 100 is indicative of the subject being sufficiently healthy to become a donor of intestinal microflora. In certain embodiments, a TCNI value of 100 is indicative of the subject being sufficiently healthy to become a donor of intestinal microflora.

The present invention further provides, in another aspect, a composition comprising at least one aerobic oncolytic bacteria, wherein the at least one aerobic oncolytic bacteria is obtained, isolated or derived from a healthy donor, wherein the healthy donor is tested to have a TCNI value in the range of 71 to 100 by any one of the methods described above.

The term “composition” is used herein in its broadest sense and generally is intended to encompass a product comprising the specified ingredients.

The term “aerobic oncolytic bacteria” as used herein refers to any bacteria which are obligate aerobes, facultative anaerobes, microaerophiles or aero-tolerant anaerobes, which are known or tested to be highly cytotoxic to cancer cells, particularly to human cancer cells. In certain embodiments, the aerobic oncolytic bacteria are known or tested to have a TCNI value in the range of 71 to 100, 76 to 100, 81 to 100, 90 to 100 or 95 to 100, or a TCNI value of 100.

In certain embodiments, the at least one aerobic oncolytic bacteria is selected from the group consisting of Escherichia coli, a Streptococcus, Enterococcus faecalis, Enterococcus faecium, and any combination thereof. In certain embodiments, the bacterial strain is Escherichia coli. In certain embodiments, the at least one aerobic oncolytic bacteria is selected from the group consisting of Escherichia coli A.T.C.C. 55373, Escherichia coli A.T.C.C. 55374, Escherichia coli A.T.C.C. 55375, Streptococcus faecalis A.T.C.C. 55376, and any combination thereof. In certain embodiments, the composition comprises Escherichia coli A.T.C.C. 55373, Escherichia coli A.T.C.C. 55374, Escherichia coli A.T.C.C. 55375, and Streptococcus faecalis A.T.C.C. 55376.

In certain embodiments, the composition is tested to have a TCNI value in the range of 71 to 100. In certain embodiments, the at least one aerobic oncolytic bacteria is tested to have a TCNI value in the range of 71 to 100. In certain embodiments, the composition further comprises at least one additional strain of bacteria, the composition thus comprising a mixture of bacteria, wherein the mixture is tested to have a TCNI value in the range of 71 to 100.

In certain embodiments, the at least one additional strain of bacteria is anaerobic bacteria. In certain embodiments, the anaerobic bacteria are selected from an anaerobic Bifidobacterium species, an anaerobic Lactobacillus species, and any combination thereof. In certain embodiments, the at least one additional strain of bacteria is selected from the group consisting of Enterococcus faecalis and Enterococcus faecium.

To support the initial growth of bacteria in the digestive tract of a subject or a patient, especially the growth of the highly-oncolytic aerobic bacteria, the composition may comprise adequate chemicals or nutrients. In certain embodiments, the composition further comprises nutritional supplements for bacteria, selected from the groups consisting of peptides, peptones, vitamins, trace elements, minerals, and any combination thereof. Each possibility represents a separate embodiment of the invention.

After being administered to the body of a subject or a patient, it is beneficial that the patient body would retain the composition at least for a minimal period of time in order for the bacteria to propagate and spread throughout the intestine. However, certain dysbiosis-associated conditions manifest in mild-to-severe diarrhea, which presents a technical obstacle to retaining the composition within the intestine. In certain embodiments, the composition further comprises a diarrhea-treating or constipation-inducing compound. The term “diarrhea-treating or constipation-inducing compound” as used herein includes any compound known to stop diarrhea, attenuate diarrhea, promote constipation and/or induce constipation with acceptable side effects, as known in the field. Many such compounds are known, such as Loperamide (4-[4-(4-Chlorophenyl)-4-hydroxypiperidin-1-yl]-N,N-dimethyl-2,2-diphenyl-butanamide), Bismuth subsalicylate (2-Hydroxy-2H,4H-benzo[d]1,3-dioxa-2-bismacyclohexan-4-one), Crofelemer, Alosetron (5-methyl-2-[(4-methyl-1H-imidazol-5-yl)methyl]-2,3,4,5-tetrahydro-1H-pyrido[4,3-b]indol-1-one), and many more.

It may be prudent to clear the patient intestine from its native microflora before administering the compositions of the present invention into a patient. In certain embodiments, the method further comprises performing an enema to the patient prior to the administration of the composition comprising a mixture of anaerobic and aerobic highly-oncolytic bacteria. It should be understood that the compositions of the present invention may be repeatedly administered to the patient until a satisfactory result is achieved. In certain embodiments, the method comprises repeated administration of the composition comprising a mixture of anaerobic and aerobic bacteria to the patient.

In certain embodiments, the TCNI value is in the range of 76 to 100, 81 to 100, 86 to 100, 90 to 100, or 95 to 100, or the TCNI value is 100. In certain embodiments, the TCNI value is in the range of 76 to 100. In certain embodiments, the TCNI value is in the range of 81 to 100. In certain embodiments, the TCNI value is in the range of 86 to 100. In certain embodiments, the TCNI value is in the range of 90 to 100. In certain embodiments, the TCNI value is in the range of 95 to 100. In certain embodiments, the TCNI value is 100.

The composition may be administered to a subject or a patient by one of several routes known in the field. Often, the most appropriate routes would be oral and rectal administration. In certain embodiments, the compositions described above may be formulated for oral administration. The term “oral administration” means the enteral administration of a dosage form commonly known as oral dosage form. Oral dosage forms are in particular solid oral dosage forms containing defined amounts of the active agent, such as capsules or sachets, but also liquid dosage forms, such as droplets, suspensions, or emulsions. Suitable excipients such as sorbitol, lactose, starch, or magnesium stearate, may be admixed. Soft capsules, may contain the liquid dosage forms mentioned, in particular suspensions or emulsions. They may contain, as additives, glycerol, lecithin, fats, oil, paraffin oil or liquid polyethylene glycol. Many bacteria-based products, probiotics included, often convey a unique sense of smell and taste, dictated by the type and by the concentration of the respective bacteria. When administered orally, both taste and smell may deter a subject from repeated or continuous use. In certain embodiments, the composition further comprises a flavorant. In certain embodiments, the composition comprises an aroma compound.

The terms “flavorant” and “aroma compound” are used in their broadest sense, and include compounds routinely used in the field of edible products to improve a product's smell and/or taste. Flavor is the sensory impression of a food or other substance, and is determined mainly by the chemical senses of taste and smell. Flavorant is defined as a substance that gives another substance flavor, altering the characteristics of the solute, causing it to become sweet, sour, tangy, etc. An aroma compound, also known as odorant, aroma, fragrance, or flavor, is a chemical compound that has a smell or odor. A chemical compound has a smell or odor when it is sufficiently volatile to be transported to the olfactory system in the upper part of the nose.

Many formulations are known to be appropriate for delivering compositions by oral administration, all of which are considered appropriate by the present invention. In certain embodiments, the composition is formulated as capsules, hard or soft gelatin capsules, pills, tablets, dragees, solutions, suspensions, liquids, gels, slurries, drops, granulates, syrups, or controlled or delayed release forms thereof. To protect the microflora of the composition from the extremely acidic conditions found in the stomach, there may be a need to coat the composition with, or entrap the composition within, an enteric coating. An enteric coating is usually a polymer barrier applied on oral medication. This barrier protects drugs from the pH (i.e. acidity) of the stomach. Most enteric coatings work by presenting a surface that is stable at the highly acidic pH found in the stomach, but breaks down rapidly at a less acidic (relatively more basic) pH. For example, they will not dissolve in the acidic juices of the stomach (pH ˜3), but they will in the alkaline (pH 7-9) environment present in the small intestine. Materials used for enteric coatings include fatty acids, waxes, shellac, plastics, and plant fibers. In certain embodiments, the composition is coated by one or more enteric coating.

Suitable carriers for oral administration are well known in the art. Compositions for oral use can be made using a solid excipient, optionally grinding the resulting mixture, and processing the mixture of granules, after adding suitable auxiliaries as desired, to obtain tablets or dragee cores. Non-limiting examples of suitable excipients include fillers such as sugars, including lactose, sucrose, mannitol, or sorbitol, cellulose preparations such as, maize starch, wheat starch, rice starch, potato starch, gelatin, gum tragacanth, methyl cellulose, hydroxypropylmethyl-cellulose, and sodium carbomethylcellulose, and/or physiologically acceptable polymers such as polyvinylpyrrolidone (PVP).

If desired, disintegrating agents, such as cross-linked polyvinyl pyrrolidone, agar, or alginic acid or a salt thereof, such as sodium alginate, may be added. Capsules and cartridges of, for example, gelatin for use in a dispenser may be formulated containing a powder mix of the compound and a suitable powder base, such as lactose or starch.

Solid dosage forms for oral administration include capsules, tablets, pill, powders, and granules. In such solid dosage forms, the active compound is admixed with at least one inert pharmaceutically acceptable carrier such as sucrose, lactose, or starch. Such dosage forms can also comprise, as it normal practice, additional substances other than inert diluents, e.g., lubricating, agents such as magnesium stearate. In the case of capsules, tablets and pills, the dosage forms may also comprise buffering, agents. Tablets and pills can additionally be prepared with enteric coatings.

Liquid dosage forms for oral administration may further contain adjuvants, such as wetting agents, emulsifying and suspending agents, and sweetening, flavoring and perfuming agents. According to some embodiments, enteral coating of the composition is further used for oral or buccal administration. The term “enteral coating”, as used herein, refers to a coating which controls the location of composition absorption within the digestive system. Non-limiting examples for materials used for enteral coating are fatty acids, waxes, plant fibers or plastics.

A more direct approach than oral administration is rectal administration, which although presumingly less convenient to the subject, is more efficient and acts faster. In certain embodiments, the composition described above is formulated for rectal administration. As used herein, the term “rectal administration” refers to those modes of administering a compound to a subject by means of insertion through the rectum. The term “rectal formulation” encompasses those pharmaceutical formulations that are suitable for the rectum. Many formulations are known to be appropriate for delivering compositions by rectal administration, all of which are considered appropriate by the present invention. In certain embodiments, the composition is formulated as an enema, a suppository, or a rectal solution. Each possibility represents a separate embodiment of the invention.

As described above, the compositions provided by the present invention comprise a unique mixture of anaerobic and aerobic oncolytic bacteria, derived from donors of exceptional health. These compositions are therefore highly suitable to overcome dysbiosis and produce a healthy intestinal microflora within subjects. In certain embodiments, the compositions described above are for use in treating a dysbiosis-related disease or condition.

The phrase “treating a dysbiosis-related disease or condition” as used herein, refers to administering a therapeutic effective amount of the composition to a patient diagnosed with a dysbiosis-related disease or condition, to inhibit the further aggravation of the disease or condition, to attenuate at least one symptom of the disease or condition, or to eliminate at least one symptom of the disease or condition. The term “therapeutically effective amount” refers to an amount of a composition effective to treat a disease or disorder in a mammal. In the case of dysbiosis, the therapeutically effective amount of the drug may restore the natural healthy intestinal microflora. In certain embodiments, the disease or condition is selected from the group consisting of dysbiosis, inflammatory bowel disease, chronic fatigue syndrome, obesity, cancer and colitis. Each possibility represents a separate embodiment of the invention.

As described above, after being administered to the body of a subject or a patient, it is beneficial that the patient body would retain the composition at least for a minimal period of time in order for the bacteria to propagate and spread throughout the intestine. In certain embodiments, the method further comprises the step of administering a constipation-inducing composition comprising a diarrhea-treating or constipation-inducing compound to the patient.

The present invention further provides, in another aspect, an oral dosage form, comprising any one of the compositions described above.

The present invention further provides, in another aspect, a rectal dosage form, comprising any one of the compositions described above.

The present invention further provides, in another aspect, a pharmaceutical, probiotic or nutraceutical composition comprising any one of the compositions described above.

The term “pharmaceutical composition” as used herein refers to any composition or dosage form comprising or consisting of any one of the compositions of comprising therapeutically effective amount of at least one aerobic oncolytic bacteria, and a pharmaceutically-acceptable carrier. The term “pharmaceutically acceptable carrier” or “carrier” refers to any inert material (e.g., a diluent), organic or inorganic, that does not cause significant irritation to a subject and does not abrogate the biological activity and properties of an administered active ingredient. Suitable carriers can be selected based on the means of administration, as is understood by one skilled in the art.

The term “probiotic composition” as used herein refers to a composition comprising one or more probiotic organisms and one or more acceptable excipients suitable for application to a mammal It will be appreciated that acceptable excipients will be well known to the person skilled in the art of probiotic composition preparation. Examples of such acceptable excipients include: sugars such as sucrose, isomerized sugar, glucose, fructose, palatinose, trehalose, lactose and xylose; sugar alcohols such as sorbitol, xylitol, erythritol, lactitol, palatinol, reduced glutinous starch syrup and reduced glutinous maltose syrup; polysaccharides as maltodextrins, starches like maize starch, rice starch, potato starch and wheat starch, emulsifiers such as sucrose esters of fatty acid, glycerin esters of fatty acid and lecithin; thickeners (stabilizers) such as carrageenan, xanthan gum, guar gum, pectin and locust bean gum; acidifiers such as citric acid, lactic acid and malic acid; fruit juices such as lemon juice, orange juice and berry juice; vitamins such as vitamin A, vitamin B, vitamin C, vitamin D and vitamin E; and minerals such as calcium, iron, manganese and zinc.

The term “nutraceutical composition” refers to any substance that is a food or a part of a food and provides medical or health benefits, including the prevention and treatment of disease or disorder.

The present invention further provides, in another aspect, any one of the compositions described above for use in treating a dysbiosis-related disease or condition in a patient.

The term “dysbiosis-related disease or condition” as used herein refers to any disease or condition causing, or caused by, a non-healthy composition of intestinal bacteria.

The term “therapeutically effective amount” refers to an amount of a composition effective to treat a disease or disorder in a mammal. In the case of dysbiosis, the therapeutically effective amount of the bacteria or mixture of bacteria may restore the natural healthy intestinal microflora. In certain embodiments, the dysbiosis-related disease or condition is selected from the group consisting of dysbiosis, cancer, inflammatory bowel disease, chronic fatigue syndrome, obesity and colitis. Each possibility represents a separate embodiment of the invention. In certain embodiments, the dysbiosis-related disease or condition is dysbiosis.

In certain embodiments, the dysbiosis-related disease or condition is cancer. In certain embodiments, the cancer is selected from brain, lung, pancreatic and prostate cancer. In certain embodiments, the cancer is brain cancer. In certain embodiments, the cancer is lung cancer. In certain embodiments, the cancer is pancreatic cancer. In certain embodiments, the cancer is prostate cancer. In certain embodiments, the cancer is stage II cancer. In certain embodiments, the cancer is stage IV cancer. In certain embodiments, the cancer is a solid cancer. In certain embodiments, the cancer is metastatic. In certain embodiments, the cancer is non-metastatic. In certain embodiments, the patient had not received any previous anti-cancer treatment. In certain embodiments, the patient had received any previous anti-cancer treatment. In certain embodiments, the previous anti-cancer treatment is radiation therapy or chemotherapy. In certain embodiments, the previous anti-cancer treatment is radiation therapy and chemotherapy.

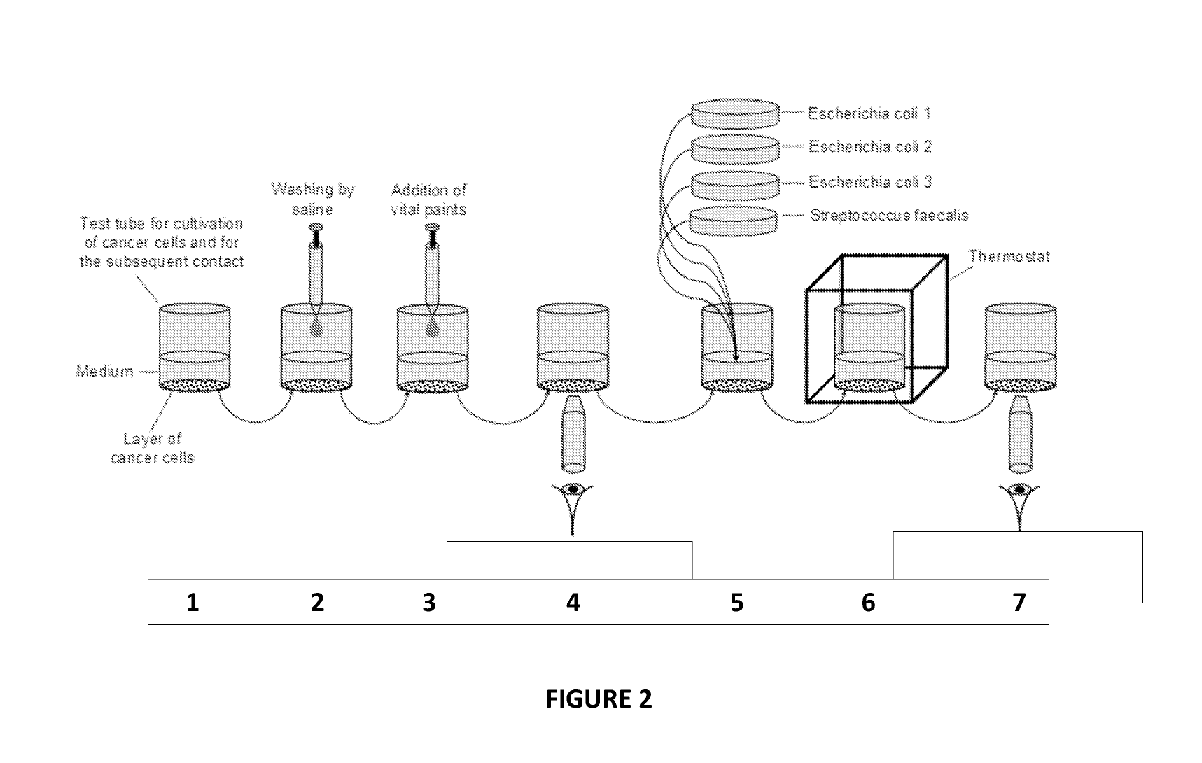

In certain embodiments, the methods describe above comprise the steps illustrated in FIG. 2. In certain embodiments, the method comprises or consists the steps of (i) cultivation of cancer cells in a culture vessel, (ii) flushing the culture vessel with saline for removing antibiotics, (iii) adding vital dye, (iv) calculating the number of cancer cells in view before contact with aerobic bacteria by an inverse microscope, or its computer analog, (v) inoculation of aerobic human intestines flora in the culture vessel together with the cancer cells, (vi) incubating the culture vessel in a thermostat for aerobic microflora of intestines and cancer cells to contact, and (vii) calculating the number of cancer cells in view after contact with the aerobic bacteria by an inverse microscope, or its computer analog.

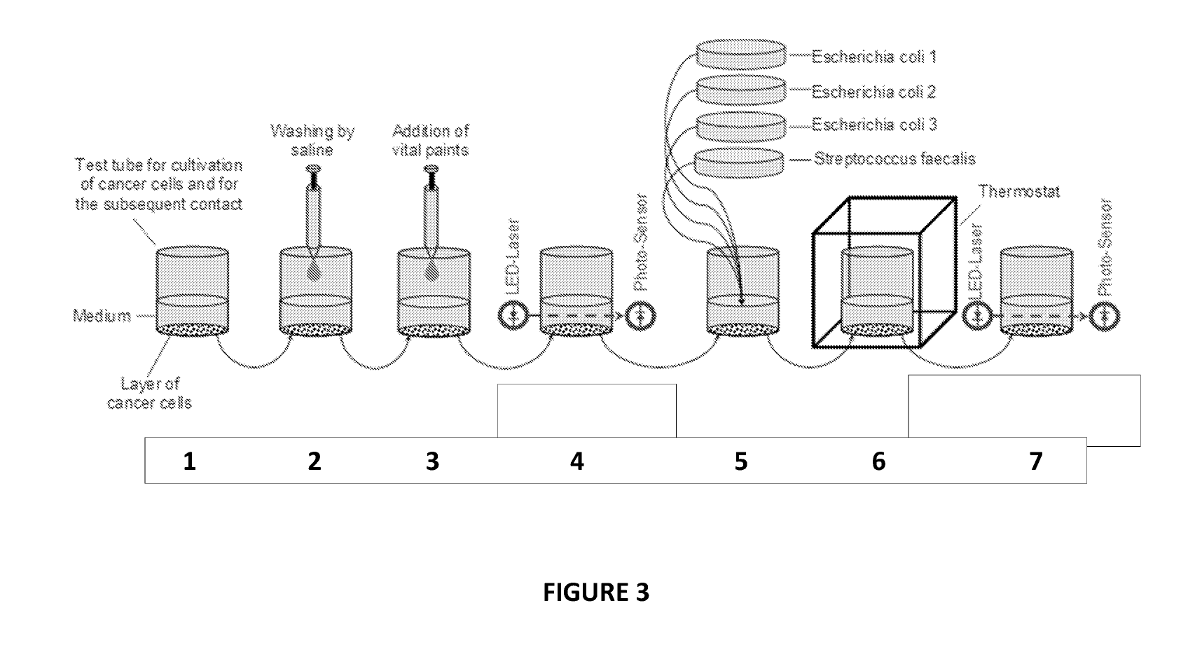

In certain embodiments, the methods describe above comprise the steps illustrated in FIG. 3. In certain embodiments, the method comprises or consists the steps of (i) cultivation of cancer cells in a culture vessel, (ii) flushing the culture vessel with saline for removing antibiotics, (iii) adding vital dye, (iv) measuring the concentration of intracellular contents, for instance, a cytoplasmic protein, by laser spectrophotometer before contact with aerobic bacteria, (v) inoculation of aerobic human intestines flora in the culture vessel together with the cancer cells, (vi) incubating the culture vessel in a thermostat for aerobic microflora of intestines and cancer cells to contact, and (vii) measuring the concentration of intracellular contents, for instance, a cytoplasmic protein, by laser spectrophotometer after contact with the aerobic bacteria.

In some embodiments, the numbers expressing quantities of ingredients, properties such as molecular weight, reaction conditions, and so forth, used to describe and claim certain embodiments of the application are to be understood as being modified in some instances by the term “about”. Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable. In some embodiments, the term “about” means a deviation of 10% of the indicated value.

The following examples are presented in order to more fully illustrate some embodiments of the invention. They should, in no way be construed, however, as limiting the broad scope of the invention.

### EXAMPLES

### Example 1

### Direct Evaluation of Cancer Cells' Viability, Prior and Post the Addition of Bacteria

Standardized human cancer cells were inoculated into and grown in a sterile culture vessel (FIG. 2). Then, the cells' medium was aseptically removed from the culture vessel, cells were briefly washed by saline, and added a dye capable of differentially dying intact and raptured cells. Cell viability dyes such as the tetrazolium dye MTT, which labels live cells, and Trypan blue, which labels dead cells, were used alone or in combination to determine the number of intact or raptured cells, and/or their respective ratio. After dying was complete, the number of cancer cells (dead, alive and/or total) in the culture vessel (before contact with a sample of control or test bacteria) was determined electronically by a digital camera and an image analyzer. Then, a sample of standardized bacteria such as aerobic bacteria of the normal intestinal microflora, such as Escherichia coli and any species of Streptococcus was inoculated into the sterile culture vessel (about 3×10⁶ bacteria per a cancer cell), and allowed sufficient time (2-6 hours) under adequate conditions to contact the cancer cells. After sufficient contact was achieved, the number of cancer cells (dead, alive and total) in the culture vessel was once again determined visually either manually or electronically.

### Example 2

### Indirect Evaluation of Cancer Cells' Viability, Prior and Post the Addition of Bacteria

Standardized human cancer cells were inoculated into and grown in a sterile culture vessel (FIG. 3). Then, the cells' medium was aseptically removed from the culture vessel, cells were briefly washed by saline, and added a dye capable of differentially dying intact and raptured cells. Cell viability dyes such as Calcein AM, which labels live cells, and Fixable Viability Dye eFluor® 455UV, which labels dead cells, were used alone or in combination to determine the number of intact or raptured cells, or their respective ratio. After dying was complete, the number of cancer cells (dead, alive and/or total) in the culture vessel (before contact with a sample of control or test bacteria) was determined electronically by a spectrophotometer. Then, a sample of standardized bacteria such as aerobic bacteria of the normal intestinal microflora, such as Escherichia coli and any species of Streptococcus was inoculated into the sterile culture vessel (about 3×10⁶ bacteria per a cancer cell), and allowed sufficient time (2-6 hours) under adequate conditions to contact the cancer cells. After sufficient contact was achieved, the number of cancer cells (dead, alive and total) in the culture vessel was once again determined electronically.

### Example 3

### Clinical Evaluation of the Distribution of TCNI in Populations of Healthy Subjects and Cancer Patients

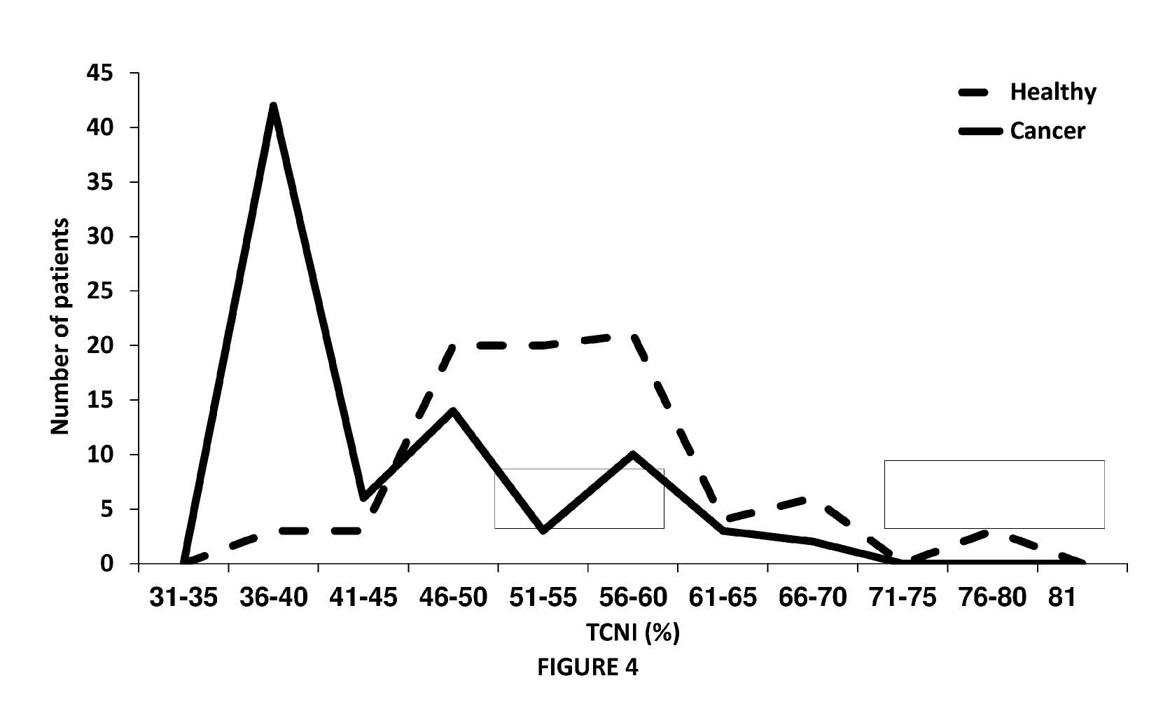

80 healthy volunteers and 80 cancer patients were enrolled to a clinical study at Tel-HaShomer hospital, Israel, and the TCNI value for each subject was determined using the methods described above. Table 1 summarizes the results obtained. FIG. 4 graphically depicts the results of the study. As can be seen, 60% of the cancer patients had a TCNI value below 46, while only 7.5% of the healthy subjects had similar TCNI values. On the other hand, no cancer patients had a TCNI value over 70, while about 4% of the healthy subjects had TCNI values over 70. Of importance, 60% of the cancer patients had a TCNI value below 46, about 80% of the cancer patients had a TCNI value below 56, 97.5% of the cancer patients had a TCNI value below 66, and not a single cancer patient out of 80 single cancer patients had a TCNI value over 71.

TABLE 1

| | | % of the | | % of the |
|---|---|---|---|---|
| TCNI | # of heathy | total number of | # of cancer | total number |
| (%) | subjects | healthy subjects | patients | of cancer patients |
| 0-35 | 0 | 0 | 0 | 0 |
| 36-40 | 3 | 3.75 | 42 | 52.5 |
| 41-45 | 3 | 3.75 | 6 | 7.5 |
| 46-50 | 20 | 25 | 14 | 17.5 |
| 51-55 | 20 | 25 | 3 | 3.75 |
| 56-60 | 21 | 26.25 | 10 | 12.5 |
| 61-65 | 4 | 5 | 3 | 3.75 |
| 66-70 | 6 | 7.5 | 2 | 2.5 |
| 71-75 | 0 | 0 | 0 | 0 |
| 76-80 | 3 | 3.75 | 0 | 0 |
| 81-100 | 0 | 0 | 0 | 0 |
| Total | 80 | 100 | 80 | 100 |

### Example 4

### Double Blind, Clinical Evaluation of Healthy Subjects and Cancer Patients

103 healthy volunteers and 111 cancer patients were enrolled to a clinical study at Tel-HaShomer hospital, Israel, and the TCNI value for each subject was determined using the methods described above. A TCNI value of 40 was determined to be the threshold below which a patient is diagnosed positive for cancer, and above which a patient is diagnosed as negative for cancer (healthy). Table 2 summarizes the results obtained. Using a threshold TCNI value of 40, the diagnosis method achieved 79% sensitivity, 78% specificity and 79% precision.

TABLE 2

| | | % of the total | | % of the total | |
|---|---|---|---|---|---|
| | # of true | number of | # of false | number of | |
| | positives | cancer patients | negative | cancer patients | Total |
| Cancer | 88 | 79 | 23 | 21 | 111 |
| patients | | | | | |
| | | % of the total | | % of the total | |
| | # of false | number of | # of true | number of | |
| | positives | cancer patients | negative | cancer patients | Total |
| Healthy | 23 | 22 | 80 | 78 | 103 |
| subjects | | | | | |

### Example 5

### Treatment of Cancer Patients with Probiotics Derived from Health Subjects

Eight cancer patients in different stages of the disease were treated with a 2 gram lyophilized probiotic product, comprising Escherichia coli A.T.C.C. 55373, Escherichia coli A.T.C.C. 55374, Escherichia coli A.T.C.C. 55375, Streptococcus faecalis A.T.C.C. 55376, 3 times a day for a period of 3 months. 150 apparently healthy volunteers were screened by the methods provided herein, and bacteria were isolated from a healthy subject having a TCNI of about 95%. Table 3 summarizes patients' details, previous treatments, condition upon initiating probiotic therapy and outcome of probiotic therapy.

TABLE 3

| Patient | Patient | Previous | Condition upon initiating | |
|---|---|---|---|---|
| # | details | treatment(s) | probiotic therapy | Probiotic therapy outcome |
| 1 | G. B., | Radiation | Left lung: stage IV | Left lung: a cyst (after 4 |
| | 64 y, Italy | therapy | Brain: 4 metastases | months) |
| | | | | Brain: 3 vanished, 1 became |
| | | | | a cyst (after 1 month) |
| | | | | Died of myocardial |
| | | | | infarction 2 years later |
| 2 | S. B., | None | Right lung: stage II | Right lung: no tumor (after 4 |
| | 66 y, | | Metastases: 2 years after | months) |
| | Israel | | colon resection | Alive. |
| 3 | S. N., | Radiation | Brain: stage IV | Brain: no tumor (after 4 |
| | 72 y, | therapy | | months) |
| | France | | | Alive. |
| 4 | A. N., | Chemo- | Pancreas: stage IV | Pancreas: a cyst (after 6 |
| | 48 y, | therapy | Liver: metastases | months) |
| | Israel | | | Liver: no metastases (after 6 |
| | | | | months) |
| | | | | Died of myocardial |
| | | | | infarction 2 years later |
| 5 | S. R., | Chemo- | Prostate: stage IV | Prostate: a cyst (after 4 |
| | 69 y, | therapy | Pelvis: multi-metastases | months) |
| | Israel | | | Pelvis: no metastases (after 4 |
| | | | | months) |
| | | | | Died 3 year later of cerebral |
| | | | | hemorrhage |
| 6 | A. S., | None | Prostate: stage III | Prostate: a cyst (after 4 |
| | 72 y, | | Lymph nodules: | months) |
| | Israel | | metastases | Lymph nodules: no |
| | | | | metastases (after 4 months) |
| | | | | Alive |
| 7 | N. B., | Radiation | Brain: stage IV | Died |
| | 39 y, | therapy | | |
| | Israel | | | |
| 8 | A. Z., | Radiation | Brain: stage IV | Died |
| | 33 y, | and chemo- | | |
| | Israel | therapy | | |

While the invention has been shown and described in detail, the foregoing description is in all aspects illustrative and not restrictive. It is therefore understood that numerous modifications and variations can be devised without departing from the scope of the invention.

1.-48. (canceled)

49. A method for determining the health status of a potential donor of intestinal microflora, comprising the steps of:
providing a culture vessel having adherent cancer cells under standard culture conditions;
(ii) determining a control baseline oncolytic level by determining the number of viable cancer cells or the number of dead cancer cells in the culture vessel of (i);
(iii) inoculating a culture vessel having adherent cancer cells under standard culture conditions with a test bacterial sample comprising intestinal aerobic bacteria derived from the potential donor;
(iv) incubating the inoculated culture vessel of (iii) under conditions sufficient to enable lysis of the adherent cancer cells by the intestinal aerobic bacteria;
(v) determining a test oncolytic level by determining the number of viable cancer cells or the number of dead cancer cells in the inoculated culture vessel of step (iv);
(vi) determining for the test bacterial sample a tumor cell necrosis index (TCNI) with the equation (A−B)/A×100=C, wherein C is the tumor cell necrosis index (TCNI), A is the number of viable cancer cells in the culture vessel of step (i) without incubation with a control oncolytic bacteria as determined in step (ii), or the number of dead cancer cells in the culture vessel of step (i) after incubation with a control oncolytic bacteria as determined in step (ii); B is the number of viable cancer cells as determined in step (v); and
(vii) determining the health status of said subject in accordance with the TCNI value determined in step (vi);
wherein the method does not comprise removal of said adherent cancer cells from the culture vessel; and wherein a TCNI value in the range of 71 to 100 is indicative of said potential donor being sufficiently healthy to become a donor of intestinal microflora.

50. The method of claim 49, wherein said culture vessel of step (iii) is said culture vessel of step (i).

51. The method of claim 50, wherein said adherent cancer cells of step (iii) are said adherent cancer cells of step (i).

52. The method of claim 49, wherein said adherent cancer cells comprise human cancer cells.

53. The method of claim 49, wherein the control baseline oncolytic level of step (ii) and the test oncolytic level of step (v) are each independently the number of the viable cancer cells in the respective culture vessel.

54. The method of claim 49, wherein the control baseline oncolytic level of step (ii) and the test oncolytic level of step (v) are each independently the number of dead cancer cells in the respective culture vessel.

55. The method of claim 49, wherein the control baseline oncolytic level of step (ii) is the number of viable cancer cells in the respective culture vessel, and the test oncolytic level of step (v) is the number of dead cancer cells in the respective culture vessel.

56. The method of claim 49, wherein the control baseline oncolytic level of step (ii) is the number of dead cancer cells in the respective culture vessel, and the test oncolytic level of step (v) is the number of viable cancer cells in the respective culture vessel.

57. The method of claim 49, wherein the conditions sufficient to enable lysis of the adherent cancer cells are calibrated by the extent of interaction between a control oncolytic bacteria and a control culture of adherent cancer cells.

58. The method of claim 49, wherein the method does not comprise a step inflicting a chemical or physical insult to the cancer cells of said culture vessel.

59. The method of claim 58, wherein the step inflicting a chemical or physical insult is selected from the group consisting of use of trypsin, use of a cell scraper, formation of a cell suspension, use of a toxic dye, centrifugation, and any combination thereof.

60. The method of claim 49, wherein the number of viable and/or dead cancer cells in said culture vessel of step (i) is determined (a) in the presence of a control oncolytic bacterial strain after co-incubation under conditions sufficient to enable lysis of the cancer cells by the control oncolytic bacterial strain, (b) after the addition of a control oncolytic bacterial strain but before the control oncolytic bacterial strain is able to lyse the adherent cancer cells, (c) after the addition of a control non-oncolytic bacterial strain, or (d) without the addition of any bacteria.

61. The method of claim 49, wherein a TCNI value in the range of 76 to 100, 81 to 100, 90 to 100 or 95 to 100, or a TCNI value of 100 is indicative of the subject being sufficiently healthy to become a donor of intestinal microflora.

62. A composition comprising at least one aerobic oncolytic bacteria, wherein the at least one aerobic oncolytic bacteria is obtained, isolated or derived from a healthy donor, wherein the healthy donor is tested to have a TCNI value in the range of 71 to 100 by the method of claim 49.

63. The composition of claim 62, wherein the composition is tested to have a TCNI value in the range of 71 to 100.

64. The composition of claim 62, further comprising at least one additional strain of bacteria, the composition thus comprising a mixture of bacteria, wherein the mixture is tested to have a TCNI value in the range of 71 to 100.

65. The composition of claim 64, wherein the at least one additional strain of bacteria is anaerobic bacteria.

66. The composition of claim 62, further comprising nutritional supplements for bacteria, selected from the groups consisting of peptides, peptones, vitamins, trace elements, minerals, and any combination thereof.

67. The composition of claim 62, wherein the TCNI value is in the range of 76 to 100, 81 to 100, 90 to 100, or 95 to 100, or wherein the TCNI value is 100.

68. A method for treating a dysbiosis-related disease or condition in a subject, comprising administering a composition according to claim 62 to said subject.
